(19) United States
(12) Patent Application Publication
(10) Pub. No.: US 2020/0043616 A1
(43) Pub. Date: Feb. 6, 2020

(54) GUIDELINE AND PROTOCOL ADHERENCE IN MEDICAL IMAGING

(71) Applicant: KONINKLIJKE PHILIPS N.V., EINDHOVEN (NL)

(72) Inventors: AXEL SAALBACH, HAMBURG (DE); TIM PHILIPP HARDER, AHRENSBURG (DE); TANJA NORDHOFF, HAMBURG (DE); RAFAEL WIEMKER, KISDORF (DE); FABIAN WENZEL, HAMBURG (DE); JENS VON BERG, HAMBURG (DE); IRINA WAECHTER-STEHLE, HAMBURG (DE)

(21) Appl. No.: 16/469,334

(22) PCT Filed: Dec. 6, 2017
(86) PCT No.: PCT/EP2017/081602

§ 371 Date: Jun. 13, 2019

Related U.S. Application Data
(60) Provisional application No. 62/435,099, filed on Dec. 16, 2016.

(51) Int. Cl. G16H 70/20; G16H 30/40; G16H 30/20

(52) U.S. Cl. CPC G16H 70/20; G16H 30/20; G16H 30/40

(57) ABSTRACT

A system includes an analytics unit (140), which compares a medical image (105) and associated information with a stored medical guideline (142), and identifies an error or a deviation (340) from the medical guideline based on the comparison.

### FIELD OF THE INVENTION

The following generally relates to medical imaging, and more specifically to monitoring and reporting of adherence to medical imaging guidelines and imaging protocols in healthcare provider organizations.

### BACKGROUND OF THE INVENTION

Healthcare provider organizations, such as hospitals, clinics, and the like, provide medical imaging of patients, often with different imaging modalities, such as computed tomography (CT), magnetic resonance imaging (MR), positron emission tomography (PET), single proton emission computed tomography (SPECT), ultrasound (US), combinations thereof and the like. The medical imaging of patients can include multiple modalities and imaging devices or scanners from multiple vendors including multiple vendors for scanners of a same modality.

Medical imaging of patients is governed by organizational clinical guidelines, which are derived in parts from industry medical standards, regulatory compliance, and organizational policies and procedures. The guidelines consider both the appropriateness and the economic efficiency of generation and delivery of the medical imaging. The appropriateness relates to the benefits and harms of the examination given a specific medical indication. The guidelines, in response to an ordered imaging procedure, identify an appropriate imaging protocol for proper acquisition of patient imaging data by a specific scanner or type of scanner, and image reconstruction procedures from the acquired patient imaging data. The guidelines can further specify routing procedures for the reconstructed image(s).

For example, in response to an ordered medical procedure by a physician, a CT chest image of a patient is generated of a patient by a CT technician using a CT scanner according to a governing guideline. The generated CT chest image is formatted in a Digital Imaging and Communications in Medicine (DICOM) standard format. Using the DICOM protocol, the CT chest image is transmitted to a diagnostic workstation for immediate analysis by a radiologist and/or to a Picture Archiving and Communication Systems (PACS), for later retrieval and analysis.

The imaging protocols specify scan parameter settings for acquisition by each scanner. For example, in a chest-abdomen-pelvis CT imaging protocol, scan protocol parameters include an energy level of x-ray radiation produced by a CT scanner to scan the patient, and the scan energy level parameter together with other scan parameters define a dose of x-ray radiation that is given to the individual patient during the imaging procedure. Scan parameters can be specific to a model and/or manufacturer of the scanner. The imaging protocols can include other aspects of medical imaging, such as administration of contrast agents, patient positioning, safety procedures, and the like.

Errors or deviations from guidelines can increase cost, create incorrect or poor quality images, cause repeat image data acquisition, delay time critical delivery of patient medical images, give patients unnecessary and/or additional dose of radiation, combinations thereof, and the like. For example, an error in a CT scan parameter of an imaging procedure can cause non-viable scan acquisition data, and repeating the imaging procedure with a corrected scan parameter to obtain viable scan acquisition data. However, the patient still receives the unnecessary dose of the imaging procedure with the scan parameter error, the patient is delayed for the corrected CT scan, and the CT scanner is unavailable for other patients during the correct scan.

Imaging guidelines typically include some flexibility to accommodate different patient physical attributes, ranges of diagnostic purposes, and different situations. Scan parameters, imaging protocols, and guidelines are dynamic and subject to continual change and improvement in response to vendor changes and/or improvements, published medical studies, organizational changes, and the like.

### SUMMARY OF THE INVENTION

Aspects described herein address the above-referenced problems and others.

The following describes a system and method that monitors and reports guideline and protocol adherence in medical imaging. DICOM data (i.e. medical images and associated information) are transmitted over a network, which are captured and analyzed, e.g. “sniffed.” In some embodiments, the DICOM data is acquired in a retrospective analysis from computer storage, such as the PACS. The transmitted medical images and associated information are captured and read at sending points, receiving points, network throughway points, and/or combinations thereof. The transmitted medical images and associated information are compared to clinical guidelines and errors and/or deviations are identified. The identified errors and/or deviations can be displayed on a display device. The display of errors and/or deviations can include retrospectively analyzed medical images and associated information. The display of errors and/or deviations can include textual and/or graphical displays. The display of errors and/or deviations can include different levels of aggregation or granularity.

In one aspect, a system includes an analytics unit, which compares a medical image and associated information with a stored medical guideline, and identifies an error or a deviation from the medical guideline based on the comparison.

In another aspect, a method includes comparing a medical image and associated information with a stored medical guideline. An error or a deviation from the medical guideline is identified based on the comparison.

In another aspect, a non-transitory computer-readable storage medium carrying instructions controls one or more processors to compare a medical image and associated information with a stored medical guideline, and identify an error or a deviation from the medical guideline based on the comparison.

These and other aspects of the invention will be apparent from and elucidated with reference to the embodiment(s) described hereinafter.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a medical imaging guideline and protocol adherence system.

FIG. 2 illustrates an example medical imaging guideline.

FIG. 3 illustrates a schematic illustration of an example display of a guideline error and/or deviation display.

FIG. 4 flowcharts an embodiment of a method of monitoring and reporting medical imaging guideline/protocol errors and/or deviations.

### DETAILED DESCRIPTION OF EMBODIMENTS

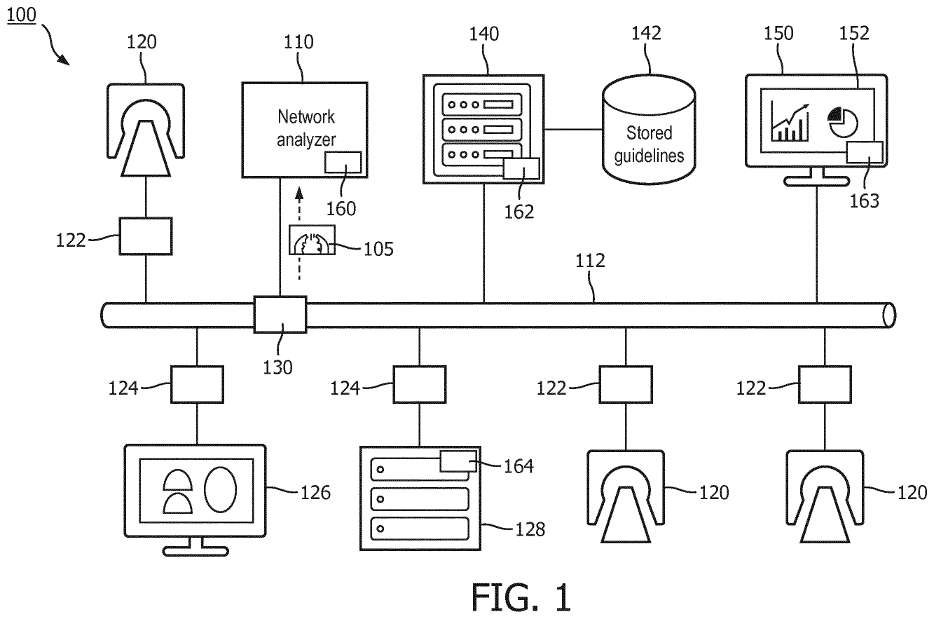

With reference to FIG. 1, an embodiment of a system 100 configured for implementing medical imaging guideline and protocol adherence is schematically illustrated. A network analyzer 110 captures and reads transmitted medical images 105 and associated information. The network analyzer 110 captures the transmitted medical images 105 and associated information using packet and/or message analysis of communication traffic on a communications network 112. The communications network 112 can include wired and/or wireless communication, cellular and/or data communication, local and/or public communication, and combinations thereof. The medical images 105 and associated information can be captured and read through intercepted messages containing the medical images and associated information, such as through the DICOM network protocol.

The network analyzer 110 can capture and read DICOM messages at sending points 122 on the network 112, such as from medical imaging devices or scanners 120. The network analyzer can capture and read DICOM messages at receiving points 124 on the network 112, such as diagnostic workstations 126 and/or image storage subsystems 128, which can include the Picture Archiving and Communication Systems (PACS), a departmental radiology information system (RIS), hospital information systems (HIS), an electronic medical record (EMR) systems, combinations thereof, and the like. The network analyzer can capture and read DICOM messages at network throughway points 130 on the network 112, such as network switches, routers, combinations thereof, and the like.

The medical images 105 and associated information are generated by the medical imaging devices 120 and include one or more modalities, such as computed tomography (CT), magnetic resonance (MR), positron emission tomography (PET), single proton emission computed tomography (SPECT), ultrasound (US), combinations or hybrids thereof, and the like. In some instances by capturing the transmitted medical images and associated information allows analysis of the medical images 105 and associated information from a plurality of modalities, a plurality of medical imaging device implementations, a plurality of medical imaging device vendors, combinations thereof, and the like. In some instances, the capture of medical images and associated information from image storage subsystems 128 allow respective analysis of medical images and associated information.

Capturing and reading the medical images 105 and associated information differs from another approach to reduce errors in imaging procedures by enforcing prospectively proper selection of scan parameters during the imaging procedure. In the prospective enforcement approach during configuration of the imaging device or scanner, an operator enters or selects information according to a predetermined process, which does not permit deviations. However, this approach suffers from being determined by implementation constraints according to imaging device software of each individual supplier of the imaging device 120. Enforcement of particular scan parameters, specific protocol selection, or specific guideline steps using software on a single imaging device may not provide the capability needed by a healthcare organization. For example, a particular scan parameter may be different for imaging device A by vendor C than imaging device B by vendor D. A new parameter is added to imaging device A, but is not available for imaging device B.

Moreover, this prospective enforcement approach does not facilitate monitoring quality across the breadth of imaging modalities, imaging device suppliers, or across the healthcare organization. That is, viewing the individual imaging devices 120 as islands of information ignores the consumption and utilization of the medical images 105 within the healthcare organization. For example, a patient receives imaging protocol A at location 1 of a healthcare organization according to an order by a primary physician, and then visits a specialist who orders imaging protocol A at location 2, thereby the patient receives duplicative imaging procedure A. In another example, an order for an imaging procedure is interpreted and performed as imaging protocol A at location 1, and interpreted and performed as imaging protocol B at location 2.

An analytics unit 140 compares the captured medical images 105 and associated information with stored medical guidelines 142. In some embodiments, the comparison includes comparing the content of the captured medical image 105 and the associated information with the outcome of an image processing step (e.g. a registration with an anatomical atlas, and the like), such as described in application “Device, system and method for quality assessment of medical images,” filed on Dec. 21, 2015 as U.S. provisional application 62/270,191, which is incorporated herein by reference in its entirety. In some embodiments, the analytics unit 140 retrieves further information, such as patient medical imaging scheduling information from a scheduling system; patient data, such as physical attributes used to determine scan parameters from an EMR or other clinical system; other scans for the patient from the image storage subsystem 128; logs of the imaging devices 120; and/or combinations thereof, and the like. In some embodiments, the analytics units uses DICOM data elements such as the study or series description, information from an order of the imaging procedure, a probabilistic matching of medical image 105 and associated information, combinations thereof, and the like as a key to identify the corresponding stored medical guideline 142.

The analytics unit 140 identifies errors or deviations from the stored medical guidelines 142. Errors or deviations can include one or more of an incorrect imaging protocol selection, an incorrect scan parameter, a repeated or a missing scan data acquisition for a patient, an incorrect image reconstruction procedure, an incorrect patient positioning, an incorrect routing procedure for the reconstructed image(s), a repeated imaging scan procedure, and the like. The analytics unit 140 searches for each type of error or deviation, and can identify one or more error or deviation for each transmitted medical image 105 and associated information.

For example, by comparing the reconstructed image with a reference image or atlas, errors or deviations between patient anatomy actually imaged and anatomy to be imaged according to a selected guideline are identified. Associated information from the DICOM data of stated anatomy imaged can be used to confirm and/or extend the analysis. Examples of errors or deviations can include incomplete anatomical imaging (not all anatomical regions according to atlas are present in the actual image), imaged regions exceed imaging protocol (image includes regions in excess of protocol), incorrect alignment of image volume or area or incorrect patient positioning (volume or area offset greater than a predetermined threshold amount, or rotational degree from a specified view plane greater a predetermined threshold amount), incorrect reconstruction (orientation of reconstructed images incorrect, slice thickness incorrect, incorrect filter applied or filter absent in reconstruction, or incorrect reconstruction algorithm employed), incorrect contrast or contrast absent (based on presence, absence, or location of contrast in image or stated in DICOM data), incorrectly timed contrast administration (contrast in motion based image in washout phase, or prior to peak uptake in targeted anatomical region). In some embodiments, errors or deviations in individual scan parameters can be identified from specific DICOM data elements or inferred from the comparison of the image with the reference image or atlas. For example, a DICOM field includes a pitch of a helical scan, which is different from the guideline.

In another example, a selected imaging protocol stated in a DICOM data element is compared with the guideline. In the absence of an imaging protocol stated within the associated information, other DICOM fields and/or information from the comparison of the image content with the atlas can be used to infer an incorrectly selected protocol, and/or deviations from the protocol according to the guideline.

In another example, an incorrect routing procedure for the reconstructed image(s) is identified from routing information of the captured and read image (105), such as the DICOM message destination, TCP/IP or OSI protocol message or packet header information, and the like. The destination addresses can be compared with the routing information 250 of the selected guideline 200. Domain name services (DNS) can be used to convert different representations of the destination presentations to a standardized format for comparison.

In some embodiments, errors and or deviations between the medical image 105 with associated information and the selected guideline can be identified and/or confirmed with additional information from other systems. For example, DICOM data elements or the image content includes identifying information that provides a timestamp of the imaging procedure and patient identification. The timestamp and patient identification used in combination with logs from a scanner can be used to identify data acquisitions performed in excess of the selected guideline. That is, the timestamp and patient identification can be used as search parameters of a particular log to identify all acquisitions for a patient within a predetermined time of the image data acquisition, and identify those which exceed the selected guideline as deviations and/or errors.

In another example, for repeated imaging procedures the timestamp and identification can be used to search for other read and captured images for the same patient within a predetermined time interval. The search can be extended to other systems, such as imaging procedure scheduling systems, EMR systems, and the like, which either provide information of planned imaging procedures and/or previously performed imaging procedures for a patient.

The analytics unit 140, by capturing and reading the medical images 105 and associated information, provides flexibility between scan parameters that differ between imaging devices and/or vendors. For example, for a CT chest imaging procedure, scan parameters may vary for imaging device A by vendor C and imaging device B by vendor D. Both sets of scan parameters, actually used in the production of the captured and read medical images 105, can be evaluated against a guideline retrospectively to determine whether each is according to guideline. As new imaging devices are added to an organization and/or new imaging procedures added to particular imaging devices, the analytics unit 140 will automatically identify errors and/or deviations according to the captured and read medical images 105 without involvement in a new imaging device and/or new imaging protocol set-up, such as in a prospective enforcement approach. That is, analysis is performed using the output of the medical images 105 by the image devices and in some instances with further information, and not in reliance of the imaging device prospective enforcement configurations. Furthermore, set-up and maintenance efforts to prospectively enforce scan parameters at an imaging device, where particular imaging procedures are not performed, are eliminated. That is, valuable personnel time is not expended to ensure rigid prospective enforcement, which may be in conflict with ability to accommodate various patient needs and imaging procedures, and additionally may not even be used.

The analytics unit 140 provides for monitoring of quality across different imaging modalities, imaging device suppliers, and/or across various organization units of a healthcare organization. For example, the captured and read medical images 105 can include new sources, such as new locations, imaging devices, and the like, which are included in the comparison with the stored medical guidelines 142. That is, as the new sources output the medical images 105, the network analyzer 110 automatically captures the additional output medical images 105, which are in turn analyzed by the analytics unit 140. The network analyzer 110 is not dependent upon access to the imaging device configuration. In another example, a duplicate imaging procedure can be identified based on two captured and read medical images 105 of the same patient and same anatomy. That is, a prospective enforcement of scan parameters would permit each procedure independently. With the captured and read medical images 105, the analytics unit 140 compares the images from each procedure and determines that they are of the same patient same anatomy, both within a predetermined time interval to be considered as duplicative or a deviation from the stored medical guidelines 142.

A dashboard unit 150 displays the identified errors and/or deviations on a display device 152. The display of errors and/or deviations can include retrospectively analyzed medical images and associated information. For example, the analyzed medical images can be previously transmitted and stored in the imaging storage subsystem, and retrieved by the network analyzer 110. The display of errors and/or deviations can include textual and/or graphical displays. The display of errors and/or deviations can include different levels of aggregation or granularity.

The network analyzer 110 is suitably embodied by one or more processors 160, such as a digital processor, a microprocessor, an electronic processor, an optical processor, a multi-processor, a distribution of processors including peer-to-peer or cooperatively operating processors, client-server arrangement of processors, and the like, communicatively connected to the network 112 and configured to capture and read traffic from the network 112. The analytics unit 140 and the dashboard unit 150 are suitably embodied by one or more configured processors 162, 163 configured to perform the disclosed comparison of the medical images 105 and associated data with the stored medical guidelines 142, identification of errors and/or deviations, and display of errors and/or deviations, respectively. The configured processor(s) 160, 162, 163, 164 execute at least one computer readable instruction stored in computer readable storage medium, such as an optical disk, a magnetic disk, semiconductor memory of a computing device with the configured processor, which excludes transitory medium and includes physical memory and/or other non-transitory medium to perform the disclosed capture and read traffic from the network 112, comparison of the medical images 105 and associated data with the stored medical guidelines 142, identification of errors and/or deviations, and display of errors and/or deviations techniques. The configured processor may also execute one or more computer readable instructions carried by a carrier wave, a signal or other transitory medium. The lines between components represented in the diagram represent communications paths.

The stored medical guidelines 142 and the image storage subsystem 128 are suitably embodied by computer storage media, such as local disk, cloud storage, remote storage, and the like, accessed by one or more configured computer processors 164, 162. The display device 152 is suitably embodiment by a computer display, projector, body worn display, and the like.

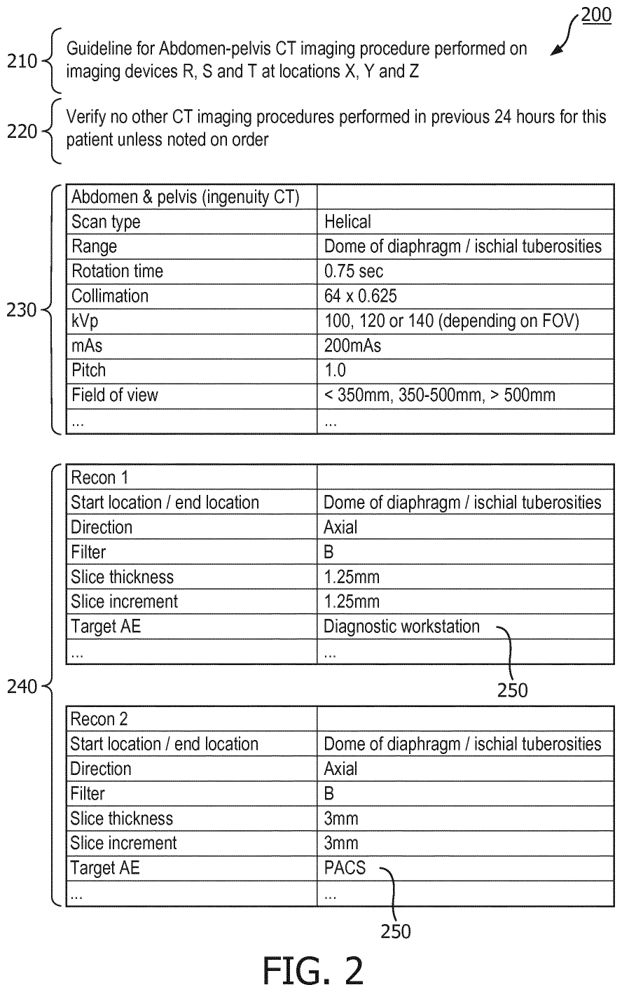

With reference to FIG. 2, an example medical imaging guideline 200 is illustrated. The medical imaging guideline 200 includes identifying information 210 specifying a scope of the guideline 200, such as areas of anatomy and modality. The identifying information can be used in identifying which guideline is compared with the captured and read medical image 105 and associated information. The identifying information 210 can include specific imaging devices, types or models of imaging devices, and the like. The identifying information 210 can include specific locations where the imaging protocol is performed.

The guideline 200 can include imaging protocol procedural information 220, such as verification procedures, patient safety procedures and set-up, contrast agent selection and preparation, and the like.

The guideline 200 include imaging protocol scan parameters 230, such as scan type, rotation time, collimation, filtering, coverage, energy level, pitch, dose, resolution, and/or combinations thereof and the like. In some embodiments, the guideline 200 includes multiple sets of scan parameters 230, each set specific to a portion of anatomy for image data acquisition.

The guideline 200 includes one or more imaging protocol reconstruction procedures 240. The reconstruction procedures 240 specify the reconstruction algorithm, acquisition data to be reconstructed, type of filters, dimensional information, and/or combinations thereof and the like.

The guideline 200 can include routing information 250. For example, routing can include addresses (electronic or physical) of one or more diagnostic workstations 126 and/or image storage subsystems 128.

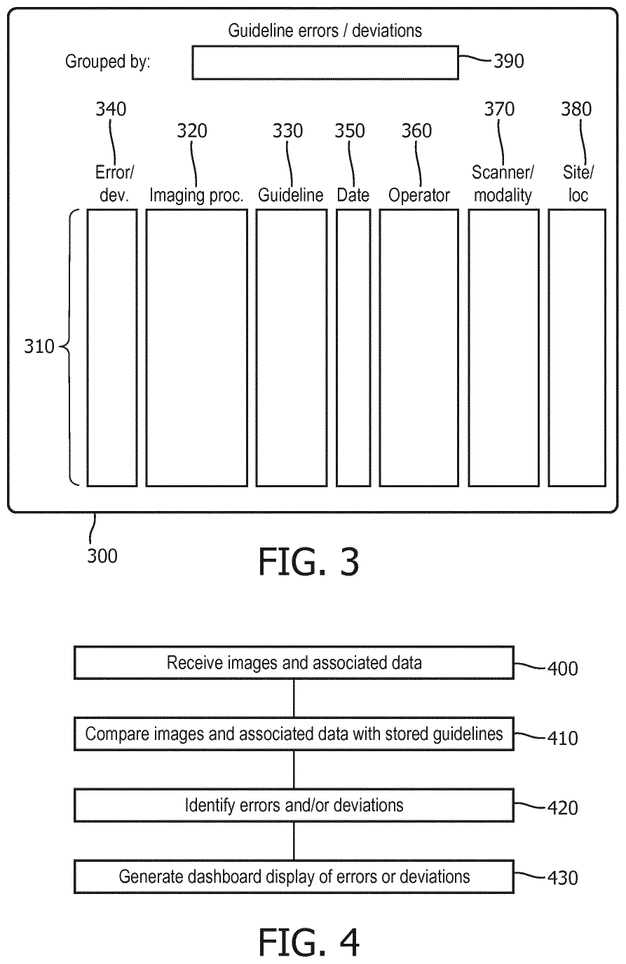

With reference to FIG. 3, a schematic illustration of an example display of a guideline error and/or deviation display 300 is illustrated. The display 300 schematically illustrates instances of guideline errors and/or deviations 310 in a list or tabular format. The display 300 can include a graphical format of guideline errors and/or deviations 310, such as pie charts, histograms, scatter plots, bar charts, and the like.

The display 300 lists ordered patient imaging procedures 320 and corresponding guidelines 330 that are matched with the patient imaging procedures 320. For each imaging procedure 320 and corresponding guideline 330, a guideline error or deviation 340 or a representation thereof is indicated.

The display 300 can include additional information, such as a date/time stamp 350 indicating when the imaging procedure 320 was performed, an operator identifier 360 of an operator performing the imaging procedure 320, an imaging device 120 identifier or modality identifier 370 identifying the device and/or modality used to perform the imaging procedure 320, a site or location identifier 380 indicating where the imaging procedure 320 was performed, and the like.

The display 300 can be ordered, selective to, or grouped by one or more of above attributes of the imaging procedure 320 or guideline 330, and used indicate the order, selection or grouping 390. For example, the display can be selective to operator Bob Jones, which indicates guideline errors and/or deviations for imaging procedures performed by Bob Jones. In another example, the display can be selective to guideline errors for imaging procedures performed using a CT modality.

The grouping can include summary information or descriptive statistics according to one or more of the above attributes. For example, a frequency of errors by type of error can be listed. In another example, an average number of errors can be listed according to site.

The summary information can be incorporated into graphical displays of the attributes or descriptive statistics of the attributes, such as number, average, mean, median, maximum, minimum, standard deviation, and the like displayed graphically.

With reference to FIG. 4, an embodiment of a method of monitoring and reporting medical imaging guideline/protocol errors and/or deviations is flowcharted.

At 400, transmitted medical images 105 and associated information are captured and read. The transmitted medical images 105 and associated information, such as transmitted in DICOM messages are captured and read from sending points 122, receiving points 124, and/or throughway points 130 on a network; image storage subsystems 128; and/or combinations thereof.

At 410, the transmitted medical images 105 and associated information are compared with the stored medical guidelines 142. The comparison includes selection of a guideline for comparison based on the medical image and associated information. The selection can include use of other systems, such as EMR, clinical systems, scheduling systems, imaging device control systems, imaging storage subsystems and the like.

At 420, errors and/or deviations between guidelines 142 and the transmitted medical images 105 with associated information are identified. Errors or deviations from can include one or more of an incorrect imaging protocol selection, use of an incorrect scan parameter, a repeated scan data acquisition for a patient, an incorrect image reconstruction, an incorrect patient positioning, an incorrect routing procedure for the reconstructed image(s), a repeated imaging scan procedure, and the like.

At 430, a display of the identified errors and/or deviations is displayed on a display device. The display can be textual or graphical. The display can summarize errors and/or deviations by attributes of the imaging procedure that generated and transmitted the medical image 105 and associated information and/or a governing guideline.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system, comprising:
at least one analytics processor configured to provide a comparison between a medical image with associated information and a stored medical guideline and identify at least one of an error and a deviation from the medical guideline based on the comparison.

2. The system according to claim 1, further comprising a network analyzer configured to capture and read the medical image with associated information transmitted over a network and communicate the medical image with associated information to the at least one analytics processor.

3. The system according to claim 1, further comprising at least one dashboard processor configured to display at least one of the identified error and the deviation from the medical guideline on a display device.

4. The system according to claim 1, wherein the medical image with associated information comprises a DICOM formatted image with associated DICOM data.

5. The system according to claim 1, wherein the stored medical guideline is selected from a plurality of stored medical guidelines based on the comparison between the medical image with associated information and identifying information of the medical guideline.

6. The system according to claim 1, wherein the at least one analytics processor searches for a plurality of errors or deviations and identifies at least one of the error and the deviation that includes at least one selected from a group comprised of:
an incorrect imaging protocol selection;
an incorrect scan parameter;
a repeated scan data acquisition for a patient;
a missing scan data acquisition for a patient;
an incorrect image reconstruction;
an incorrect patient positioning;
an incorrect routing for the reconstructed image; and
a repeated imaging scan procedure.

7. The system according to claim 1, wherein the medical guideline comprises one or more scan parameters for a medical imaging device.

8. The system according to claim 1, wherein the medical image with associated information comprise a previously transmitted medical image with associated information stored in a image storage subsystem analyzed retrospectively.

9. A method, comprising:
providing a comparison between a medical image with associated information and a stored medical guideline; and
identifying at least one of an error and a deviation from the medical guideline based on the comparison.

10. The method according to claim 9, further comprising capturing and reading the medical image with associated information transmitted over a network.

11. The method according to claim 9, further comprising displaying at least one of the identified error and the deviation from the medical guideline on a display device.

12. The method according to claim 9, wherein the medical image with associated information comprises a DICOM formatted image with associated DICOM data.

13. The method according to claim 9, further comprising selecting the stored medical guideline from a plurality of stored medical guidelines based on the comparison of the medical image with associated information and identifying information of the medical guideline.

14. The method according to claim 9, further comprising searching for a plurality of errors or deviations, wherein the identified error or the deviation includes at least one selected from a group comprised of:
an incorrect imaging protocol selection;
an incorrect scan parameter;
a repeated scan data acquisition for a patient;
a missing scan data acquisition for a patient;
an incorrect image reconstruction;
an incorrect patient positioning;
an incorrect routing for the reconstructed image; and
a repeated imaging scan procedure.

15. The method according to claim 11, wherein the medical guideline comprises one or more scan parameters for a medical imaging device.

16. A non-transitory computer-readable medium having at least one executable instruction stored thereon, which, when executed by at least one processor, cause the at least one processor to perform a method comprising:
comparing a medical image with associated information and a stored medical guideline; and
identifying at least one of an error and a deviation from the medical guideline based on the comparison.

17. The non-transitory computer-readable medium according to claim 16, further comprising capturing and reading the medical image with associated information transmitted over a network.

18. The non-transitory computer-readable medium according to claim 16, further comprising:
displaying at least one of the identified error and the deviation from the medical guideline on a display device.

19. (canceled)

20. The non-transitory computer-readable medium according to claim 16, further comprising:
selecting the stored medical guideline from a plurality of stored medical guidelines based on the comparison between the medical image with associated information and identifying information of the medical guideline.
